(19) United States
(12) Patent Application Publication
(10) Pub. No.: US 2020/0181328 A1
(43) Pub. Date: Jun. 11, 2020

(54) TWIN-TAIL HYDROCARBON SURFACTANTS FOR FOAM COMPOSITIONS

(71) Applicant: Amerex Corporation, Trussville, AL (US)

(72) Inventors: Timothy Clark, Trussville, AL (US); Kirtland Clark, Trussville, AL (US); Athanasios Karydas, Trussville, AL (US)

(73) Assignee: Perimeter Solutions LP, Clayton, MO (US)

(21) Appl. No.: 16/635,213

(22) PCT Filed: Aug. 2, 2017
(86) PCT No.: PCT/US2017/045147

§ 371 Date: Jan. 30, 2020

(51) Int. Cl. C08G 73/02; C07C 233/36; A62D 1/02; C08K 5/17

(52) U.S. Cl. CPC C08G 73/0293; C08G 73/0213; C08K 5/17; A62D 1/0071; C07C 233/36

(57) ABSTRACT

The present disclosure generally pertains to novel water soluble oligomeric and polymeric additives derived from oligomeric and polymeric amines; esters or halides of twin tail alkyl group comprising acids with intervening quaternary ammonium groups; optionally and preferably hydrophilic group comprising compounds capable of reacting with primary, secondary or tertiary amino groups; and optionally hydrophobic group comprising compounds. The compounds of the invention are useful as additives for aqueous film forming foam agents, and can perform as partial or complete replacements for fluorosurfactants and/or fluorinated foam stabilizers in foams.

### BACKGROUND OF THE INVENTION

Fluorosurfactants are useful for lowering the surface tension of water to as low as 15 dynes/cm in distilled, tap, or sea water; this property has made fluorosurfactants useful in certain commercial foam formulations. Useful fluorosurfactants typically have a single perfluoroalkyl hydrophobic tail and one or more hydrophilic tails separated by a linking group. Many references can be found in the art describing such “monomeric”, single perfluoro-tail surfactants and their use in aqueous foams and alcohol-resistant foam agents. Often, their usefulness is described as coming from the enhanced mobility of the relatively small molecule and the ability to rapidly migrate to the liquid-air interface. Linear molecules are often preferred for their lower surface tension properties. Such is the case for Capstone 1157 and 1183, Chemguard S-103A, S-106A, and FS-100, all linear, efficient surfactants having a single perfluoroalkyl tail and a single hydrophilic tail, and used effectively by foam manufacturers in many commercial foam agents.

The vapor sealing action of certain foam agents on polar solvents is achieved by (1) the precipitation of a polymer film from the polymer solution draining from the foam onto the polar solvent surface, and (2) the spreading of the aqueous film forming solution, draining from the foam, over the surface of the precipitated polymer film. The polymers commonly used in foam formulations are polysaccharide gums; water soluble polyamine-derived foam stabilizers comprising perfluoroalkyl groups and a variety of hydrophilic groups; or combinations of the two types of the aforementioned water soluble polymers.

Because of their unique surface properties, fluorosurfactants and fluorinated foam stabilizers have been used in extremely large volumes in industrial and military applications for decades. But in the 1990s, concerns started to develop: two synthetic chemicals, perfluorooctanoic acid (PFOA) and perfluorooctane sulfonic acid (PFOS) were detected globally in the environment, and in wildlife and humans. These acids have been shown to be persistent in the environment, to bioaccumulate in wildlife and humans, and to have toxicological properties of concern. Scientists were puzzled by the high concentration of these acids, as they are not part of most fluorochemical formulations. It was later postulated that the more complex perfluorooctyl substituted fluorochemicals are converted to PFOA and PFOS either by environmental degradation, or, if present in the human body, by enzymatic biotransformation.

In response to these environmental and health concerns, in 2006 the US Environmental Protection Agency (EPA) invited the eight major fluorochemicals manufacturers (which include US, European and Japanese companies) to participate in a global stewardship program and work toward the voluntary elimination of PFOA, and products that lead to PFOA, from emissions by 2015. Studies have found that perfluorohexanoic acid (PFHA) and perfluorobutanoic acid (PFBA), and their sulfonic acid analogs (PFHS and PFBS), demonstrate less severe health and environmental effects than their longer chain homologs. After the EPA stewardship program was presented, most companies gradually replaced the longer perfluoroalkyl chains with C6 (perfluorohexyl) based product. Recently, however, some environmental groups indicated concern that C6 based products may be more problematic than initially believed, which caused the implementation of tighter controls of C6 fluorochemicals in some countries. The possibility that C6 products may also be, sometime in the future, subject to restrictions, sparked efforts for the development of fluorine-free formulations.

### SUMMARY OF THE INVENTION

In one embodiment, the present disclosure relates to a water soluble aqueous film-forming foam additive composition comprising: a polyamine with a molecular weight between about 103 and 100,000; wherein the amino groups are substituted by a moiety comprising: a twin tail alkyl group; at least one quaternary ammonium group; and a non-amino hydrophilic group. In certain embodiments of the composition, the moiety further comprises a hydrophobic group. In certain embodiments of the composition, the amino groups are partially substituted. In certain embodiments of the composition, the amino groups are fully substituted. In certain embodiments, the composition is derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA). In certain embodiments, the composition is derived from at least one polyethyleneimine with a molecular weight between about 300 and 100,000.

In one embodiment, the present disclosure relates to a method of improving the stability of aqueous and alcohol-resistant foam compositions comprising the steps of: adding an effective amount of a water soluble aqueous film forming foam additive to an agent; wherein the additive comprises a polyamine with a molecular weight between about 103 and 100,000; and wherein the amino groups are substituted by: a twin tail alkyl group; at least one quaternary ammonium group; and a non-amino hydrophilic group. In certain embodiments, the amino groups are partially substituted. In certain embodiments, the amino groups are fully substituted. In certain embodiments, the amino groups are further substituted by a hydrophobic group. In certain embodiments, the additive is derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA). In certain embodiments, the additive is derived from at least one polyethyleneimine, wherein the at least one polyethyleneimine has a molecular weight between about 300 and 100,000.

### DETAILED DESCRIPTION

The present disclosure relates to novel water soluble oligomeric and polymeric additives derived from oligomeric and polymeric amines; esters or halides of twin tail alkyl group comprising acids with intervening quaternary ammonium groups; optionally and preferably hydrophilic group comprising compounds capable of reacting with primary, secondary or tertiary amino groups; and optionally hydrophobic group comprising compounds. The present disclosure also relates to methods of improving the stability of aqueous and alcohol-resistant foam compositions through the addition of such additives. Said additives allow the formulation of aqueous film forming agents having low surface tension and can perform as partial or complete replacements for fluorosurfactants and/or fluorinated foam stabilizers in foam formulations.

As used herein, the term “effective amount” means an amount of additive necessary to reduce the surface tension of aqueous and alcohol-resistant foam compositions.

As used herein, the term “fully substituted” with respect to amino groups means the replacement of substantially all amino groups with an alternative moiety.

As used herein, the term “partially substituted” with respect to amino groups means the replacement of less than all amino groups with an alternative moiety.

The presently disclosed additives are oligomers and polymers possessing at least one twin alkyl tail unit with intervening quaternary ammonium groups of Type 1; at least one hydrophilic unit of Type 2; and optionally at least one alkyl or substituted alkyl unit of Type 3. The general formula describes oligomers and polymers which comprise, in random distribution, m units of formula (1A), n units of formula (1B), p units of formula (2A), q units of formula (2B), r units of formula (2C), s units of formula (3A), and t units of formula (3B).

Wherein Type 1 units are defined as formulas (1A) and (1B),

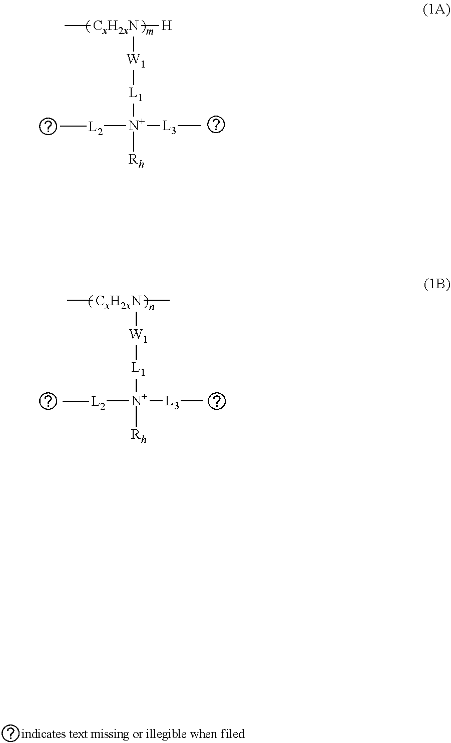

wherein: W₁ is —CO— or —SO2-; L₁ is independently a divalent linking group, either straight or branched alkylene group of 1 to 15 carbon atoms, or said alkylene group interrupted by 1 to 5 groups selected from the group consisting of —NHR₁—, —O—, —S—, —CO—, —SO₂—. —CONR₁—, —CHOH—, —NR₁CO—, —SO₂NR₁—, —NR₁SO₂—, —R₂N+R₃-, wherein R₁ is independently hydrogen or alkyl of 1 to 6 carbon atoms and R₂ and R₃ are independently alkyl groups of 1 to 6 carbon atoms; L₂, and L₃ are, independently of each other, divalent linking groups, either straight or branched alkylene groups of 1 to 10 carbon atoms, or said alkylene groups interrupted by 1 to 3 groups selected from the group consisting of —NHR₁—, —O—, —S—, —CO—, —SO₂—, —CONR₁—, —CHOH—, —NR₁CO—, —SO₂NR₁—, —NR₁SO₂—, and wherein R₁ is independently hydrogen or alkyl of 1 to 6 carbon atoms; Rₕ′ and Rₕ″ are, independently from each other, alkyl groups from 2 to 18 carbon atoms; Rₕ is an alkyl group of 1 to 6 carbon atoms; x is 2 to 6; and m and n are 0 to 500 and m+n is equal to or greater than 1.

Preferably, Rₕ′ and Rₕ″; are alkyl groups from six to twelve carbon atoms, W₁ is —CO—; L₁ is —CH₂CONH(CH₂)₃N⁺(CH₃)₂CH₂— or —CH₂—; L₂ and L₃ are, independently of each other, —CONH(CH₂)₂—, or —SO₂NH(CH₂)₂—; x is 2 or 3; n and m are 0 to 200; m+n is 2 to 200; and Rₕ is methyl.

Type 2 hydrophilic groups are defined by formulas (2A), (2B) and (2C) comprising a hydrophilic group,

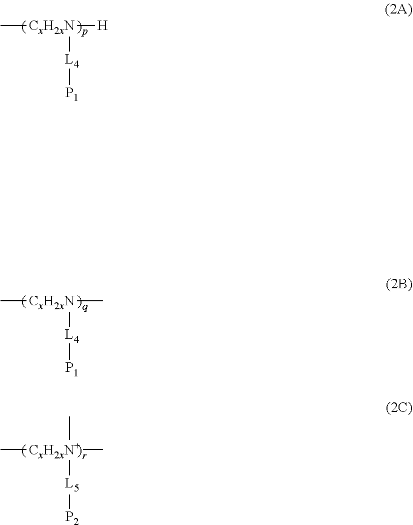

wherein: L₄ is a divalent linking group, straight or branched, saturated or unsaturated hydrocarbon group of 1 to 10 carbon atoms or said hydrocarbon group interrupted by 1 to 3 groups selected from —NHR₁—, —O—, —S—, —CONR₁—, —NR₁CO—, —SO₂NR₁—, —NR₁SO₂— or terminated with —CO—, —SO₂— where the linking group L₄ is attached to the nitrogen in formula (2A) or (2B), and wherein R₁ is independently hydrogen or alkyl of 1 to 6 carbon atoms; L₅ is alkylene with 1 to 4 carbons; P₁ is a hydrophilic group and can be —COOH, —SO₃H, —PO₃H and salts thereof, —CONH₂, —CONHCH₂OH, or —(OCH₂CH₂)ₙOH; P₂ is —COO—; x is 2 to 6; p and q are 0 to 500; and r is 1 to 200.

Preferably, L₄ is —CH₂—, —CH₂CH₂—, —CH═CH—, or —CH₂CH₂CONHC(CH₃)₂CH₂—; P₁ is —COOH, —SO₃H, or —PO₃H and salts thereof, x is 2, and p, q, and r are 1 to 100.

Type 3 alkyl and substituted alkyl units are defined as formulas (3A) and (3B),

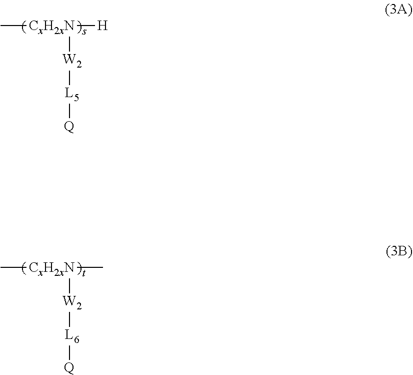

wherein: W₂ is a direct bond, —CO— or —SO₂—; L₆ is a divalent linking group, either straight or branched alkylene group of 1 to 30 carbon atoms, or said alkylene group interrupted by 1 to 3 groups selected from the group consisting —NHR₁—, —O—, —S—, —CO—, —SO₂—, —CONR₁—, —CHOH—, —NR₁CO—, —SO₂NR₁—, —NR₁SO₂—, wherein R₁ is independently hydrogen or alkyl of 1 to 6 carbon atoms; Q is hydrogen or a siloxane moiety selected from:

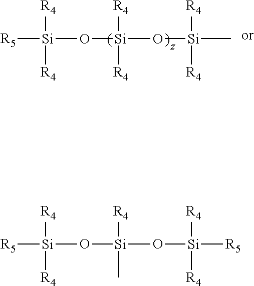

wherein R₄ and R₅ are independently lower alkyls of 1 to 6 carbon atoms and z is 1 to 100; x is 2 to 6; s and t are 0 to 200 and s+t is equal or greater than 0.

Preferably, x is 2, W₂ is —CO—, L₆ is a straight or branched alkylene group of 8 to 18 carbons, and Q is hydrogen. For some applications, s+t is most preferably equal to 0.

The presently disclosed additives can be prepared in high yields from polyamines comprising segments of formula (4A), (4B) and (4C); esters or acid halides of carboxylic and sulfonic acids of formula (5) and (6); hydrophilic reagents of formula (7A) and (7B); and hydrophobic reagents of formula (8).

Polyamines comprising segments of formula (4A), (4B) and (4C) suitable for the synthesis of the instant additives must have a combined total of at least three primary or secondary amino groups.

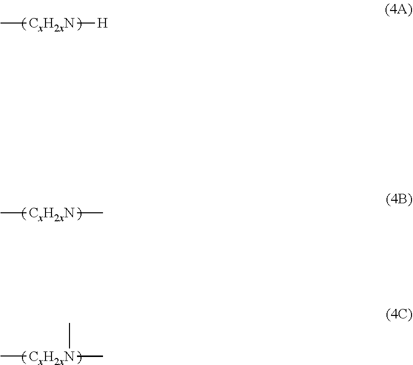

Suitable polyamines are commercially available aliphatic polyamines as described in Kirk Othmer, “Concise Encyclopedia of Chemical Technology”, John Wiley and Son, p. 350-351, (1985) and include:
Diethylenetriamine (DETA) H₂NCH₂CH₂NHCH₂CH₂NH₂
Triethylenetetramine (TETA) H₂NCH₂CH₂NHCH₂CH₂NHCH₂CHNH₂
Tetraethylenepentamine (TEPA) H₂NCH₂CH₂NHCH₂CH₂NHCH₂CH₂NHCH₂CH₂NH₂
Pentaethylene hexamine (PEHA) H₂NCH₂CH₂NHCH₂CH₂NHCH₂CH₂NHCH₂CH₂NHCH₂CH₂NH₂
Aminoethylpiperazine (AEP) NH₂CH₂CH₂N(CH₂CH₂)₂NH
Iminobispropylamine (IBPA) H₂NCH₂CH₂CH₂NHCH₂CH₂CH₂NH₂

Polyamines useful for the synthesis of the instant additives have primary, secondary, and often tertiary amines included in the polymer matrix. Higher molecular weight polyamines can be derived from the above amines, as well as from ethylene diamine, propylene diamine, 1,3-diamino propane and hexamethylene diamine, by reaction with difunctional halohydrins, or with diesters and with divinyl compounds as described in U.S. Pat. No. 2,977,245, or with haloesters.

Preferred polyamines are the polyethyleneimines or alkyl substituted polyethyleneimines, which are derived by the homopolymerization of ethyleneimine and its derivatives. Examples of such monomers yielding polyethyleneimines useful for the synthesis of the instant additives are ethyleneimine, 1,2-propyleneimine, 1,2-butyleneimine, 2,2-dimethylethyleneimine, 2,3-butyleneimine, and 2,2-dimethyl-3-n-propylethyleneimine as described in the “Journal of American Chemical Society”, Vol. 57, p. 2328 (1935) and “Journal of Organic Chemistry”, Vol. 9, p. 500 (1944).

Most important of the above polyimines are polyethyleneimines (PEIs), which are available commercially under the trade names Lupasol and Epomin, with molecular weights ranging from 300 to 100,000 and comprise approximately 25% primary amino groups, 50% secondary amino groups and 25% tertiary amino groups of formulas (4A), (4B), and (4C). Preferred PEIs have molecular weights ranging from 600 to 70,000. Depending on the properties desired, blends of PEIs with different molecular weights can also be utilized.

Alkyl groups are introduced using acids, esters, or acid halides represented by formulas (5) and (6):

Rₕ′-L₆-COOR (5)

Rₕ′-L₆-SO₂R (6)

wherein L₆ and Rₕ′ are as defined previously and R is H, alkyl, alkanol, or halide. Most important of the above esters are esters derived from carboxylic acids comprising six to twelve carbon atoms as they demonstrate lower aquatic toxicity.

Type 1 units of formulas (1A) and (1B) can be synthesized in high yields from polyamines comprising segments of formula (4A), (4B), and (4C), and the twin alkyl tail reagent shown below:

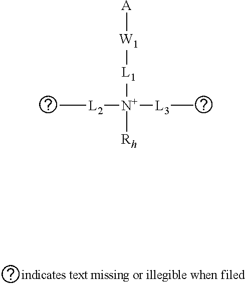

wherein Rₕ, Rₕ′, Rₕ″, W₁, L₁, L₂, and L₃ are as previously defined, and A is halogen or lower alcoxy.

Preferred twin alkyl tail reagents for use in the instant additives can be synthesized as shown below:

2 Rₕ′COOR+H₂NCH₂CH₂CH₂N(CH₃)CH₂CH₂CH₂NH₂→(Rₕ′CONHCH₂CH₂CH₂)₂N(CH₃)+ClCH₂COOCH₃→(Rₕ′CONHCH₂CH₂CH₂)₂N⁺(CH₃)CH₂COOCH₃

The above twin alkyl tail quaternary ammonium compound can be reacted with polyamines comprising segments of formula (4A), (4B) and (4C) to form units defined as formulas (1A) and (1B), or can be further chain extended with N,N-dialkylene diamines, adding a second quaternary ammonium group, followed by alkylation with a haloester, and then reacting with polyamines comprising segments of formula (4A), (4B) and (4C):

(Rₕ′CONHCH₂CH₂CH₂)₂N⁺(CH₃)CH₂COOCH₃+H₂NCH₂CH₂CH₂N(CH₃)₂→(Rₕ′CONHCH₂CH₂CH₂)₂N⁺(CH₃)CH₂CONHCH₂CH₂CH₂N(CH₃)₂+ClCH₂COOCH₃→(Rₕ′CONHCH₂CH₂CH₂)₂N⁺(CH₃)CH₂CONHCH₂CH₂CH₂N⁺(CH₃)₂CH₂COOCH₃

To prepare Type 2 hydrophilic units defined as formulas (2A), (2B), and (2C), reagents of formula (7A) and (7B) comprising a hydrophilic group as well as a group capable of reacting readily with primary and secondary amino groups, and optionally with tertiary amino groups present in polyamines comprising segments of formula (4A), (4B), and (4C) can be used.

X-L₂-P₁ (7A)

X-L₃-P₂ (7B)

wherein X is a halogen and preferably Cl; L₂, L₃, P₁, and P₂ are as previously defined.

It is well known to the skilled artisan that tertiary amino groups can be converted into betaines via carboxalkylation with halogen carboxylic acids and salts thereof, or into sulfobetaines via sulfalkylation with sultones such as propane sultone, or butane sultone.

Of the above possibilities to introduce hydrophilic groups, the methods producing segments wherein, L₂ is —CH₂—, —CH₂CH₂—, —CH═CH— or —CH₂CH₂CONHC(CH₃)₂CH₂—; P₁ is —COOH, —SO₃H, or —PO₃H and salts thereof, —CONH₂, —CONHCH₂OH or —(OCH₂CH₂)ₙOH; L₃ is alkylene with 1 to 4 carbons; and P₂ is —COO— are preferred.

To prepare Type 3 hydrophobic units of formulas (3A) and (3B), polyamines comprising segments of formula (4A), (4B), and (4C) can be reacted with reagents of formula (8) comprising a hydrophobic group and a group capable of reacting with primary and secondary amino groups.

Q-L₂-B (8)

wherein Q and L₂ are as previously defined and B is a group capable of reacting with a primary or secondary amino group.

When Q in formulas (3A) and (3B) is hydrogen, the preferred reagents of formula (8) are lower alkyl esters of long chain alkanoic acids. When Q in formulas (3A) and (3B) is a siloxane group, the preferred reagents of formula (8) are siloxanes comprising segments (9) or (10), and a group capable of reacting with primary and secondary amino groups. Most preferred are glycidyl ether terminated siloxanes of formula (11) wherein y is 10 to 80.

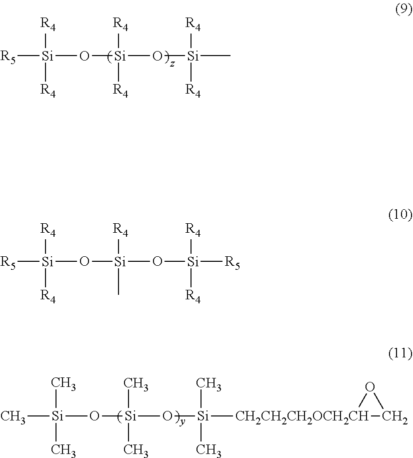

One aspect of the present disclosure is a composition comprising: a polyamine with a molecular weight between about 103 and 100,000; wherein the amino groups are substituted by a moiety comprising: at least one twin tail alkyl group; at least one quaternary ammonium group; and at least one non-amino hydrophilic group. The moiety may further comprise at least one hydrophobic group. Further, the amino groups may be either partially or fully substituted.

In particular, the amino groups may be substituted by a moiety comprising: at least two twin hydrocarbon chain units of Type 1, wherein each unit of Type 1 is selected from the group consisting of formulas (1A) and (1B), wherein: W₁ is —CO— or —SO2-; L₁ is independently a divalent linking group, either straight or branched alkylene group of 1 to 15 carbon atoms, or said alkylene group interrupted by 1 to 5 groups selected from the group consisting of —NHR₁—, —O—, —S—, —CO—, —SO₂—. —CONR₁—, —CHOH—, —NR₁CO—, —SO₂NR₁—, —NR₁SO₂—, —R₂N⁺R₃—, wherein R₁ is independently hydrogen or alkyl of 1 to 6 carbon atoms and R₂ and R₃ are independently alkyl groups of 1 to 6 carbon atoms; L₂, and L₃ are, independently of each other, divalent linking groups, either straight or branched alkylene groups of 1 to 10 carbon atoms, or said alkylene groups interrupted by 1 to 3 groups selected from the group consisting of —NHR₁—, —O—, —S—, —CO—, —SO₂—. —CONR₁—, —CHOH—, —NR₁CO—, —SO₂NR₁—, —NR₁SO₂—, and wherein R₁ is independently hydrogen or alkyl of 1 to 6 carbon atoms; Rₕ′ and Rₕ″ are, independently from each other, alkyl groups from 2 to 18 carbon atoms; Rₕ is an alkyl group of 1 to 6 carbon atoms; x is 2 to 6; and m and n are 0 to 500 and m+n is equal to or greater than 1; and at least two hydrophilic units of Type 2, wherein each unit of Type 2 is selected from the group consisting of formulas (2A), (2B) and (2C), wherein: L₄ is a divalent linking group, straight or branched, saturated or unsaturated hydrocarbon group of 1 to 10 carbon atoms or said hydrocarbon group interrupted by 1 to 3 groups selected from —NHR₁—, —O—, —S—, —CONR₁—, —NR₁CO—, —SO₂NR₁—, —NR₁SO₂— or terminated with —CO—, —SO₂— where the linking group L₄ is attached to the nitrogen in formula (2A) or (2B), and wherein R₁ is independently hydrogen or alkyl of 1 to 6 carbon atoms; L₅ is alkylene with 1 to 4 carbons; P₁ is a hydrophilic group and can be —COOH, —SO₃H, —PO₃H and salts thereof, —CONH₂, —CONHCH₂OH, or —(OCH₂CH₂)ₙOH; P₂ is —COO⁻; x is 2 to 6; p and q are 0 to 500; and r is 1 to 200. The amino groups may be further substituted by a moiety comprising: at least one alkyl or substituted alkyl unit of Type 3, wherein each Type 3 unit is selected from the group consisting of formulas (3A) and (3B), wherein: W₂ is a direct bond, —CO— or —SO2-; L₆ is a divalent linking group, either straight or branched alkylene group of 1 to 30 carbon atoms, or said alkylene group interrupted by 1 to 3 groups selected from the group consisting —NHR₁—, —O—, —S—, —CO—, —SO₂—. —CONR₁—, —CHOH—, —NR₁CO—, —SO₂NR₁—, —NR₁SO₂—, wherein R₁ is independently hydrogen or alkyl of 1 to 6 carbon atoms; and Q is hydrogen or a siloxane moiety selected from the group consisting of:

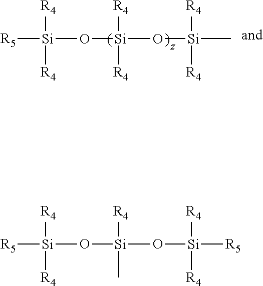

wherein R₄ and R₅ are independently lower alkyls of 1 to 6 carbon atoms and z is 1 to 100; x is 2 to 6; and s and t are 0 to 200 and s+t is equal or greater than 0.

Alternatively, the amino groups are substituted by a moiety comprising: at least two twin hydrocarbon chain units of Type 1, wherein each Type 1 unit is selected from the group consisting of formulas (1A) and (1B), wherein Rₕ′ and Rₕ″; are alkyl groups from six to twelve carbon atoms, W₁ is —CO—; L₁ is —CH₂CONH(CH₂)₃N⁺(CH₃)₂CH₂— or —CH₂—; L₂ and L₃ are, independently of each other, —CONH(CH₂)₂—, or —SO₂NH(CH₂)₂—; x is 2 or 3; n and m are 0 to 200; m+n is 2 to 200; and Rₕ is methyl; and at least two hydrophilic units of Type 2, wherein each Type 2 unit is selected from the group consisting of formulas (2A), (2B) and (2C), wherein L₄ is —CH₂—, —CH₂CH₂—, —CH═CH—, or —CH₂CH₂CONHC(CH₃)₂CH₂—; P₁ is —COOH, —SO₃H, or —PO₃H and salts thereof, x is 2, and p, q, and r are 1 to 100. In such composition, the amino groups may be substituted by a moiety further comprising: at least one alkyl or substituted alkyl unit of Type 3, wherein each Type 3 unit is selected from the group consisting of formulas (3A) and (3B), wherein x is 2, W₂ is —CO—, L₆ is a straight or branched alkylene group of 8 to 18 carbons, and Q is hydrogen.

The composition may be derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA). The composition may also be derived from at least one polyethyleneimine with a molecular weight between about 300 and 100,000.

Aqueous foams may collapse or be destroyed in the presence of polar solvents. As such, another aspect of the present disclosure is a method of improving the stability of aqueous and alcohol-resistant foam compositions comprising the steps of: adding an effective amount of a water soluble aqueous film forming foam additive to an agent; wherein the additive comprises a polyamine with a molecular weight between about 103 and 100,000; and wherein the amino groups are substituted by a moiety comprising: at least one twin tail alkyl group; at least one quaternary ammonium group; and at least one non-amino hydrophilic group. The amino groups may be substituted by a moiety further comprising at least one hydrophobic group. The amino groups may be either partially or fully substituted.

In particular, the amino groups may be substituted by a moiety comprising: at least two twin hydrocarbon chain units of Type 1, wherein each Type 1 unit is selected from the group consisting of formulas (1A) and (1B), wherein: W₁ is —CO— or —SO2-; L₁ is independently a divalent linking group, either straight or branched alkylene group of 1 to 15 carbon atoms, or said alkylene group interrupted by 1 to 5 groups selected from the group consisting of —NHR₁—, —O—, —S—, —CO—, —SO₂—. —CONR₁—, —CHOH—, —NR₁CO—, —SO₂NR₁—, —NR₁SO₂—, —R₂N⁺R₃—, wherein R₁ is independently hydrogen or alkyl of 1 to 6 carbon atoms and R₂ and R₃ are independently alkyl groups of 1 to 6 carbon atoms; L₂, and L₃ are, independently of each other, divalent linking groups, either straight or branched alkylene groups of 1 to 10 carbon atoms, or said alkylene groups interrupted by 1 to 3 groups selected from the group consisting of —NHR₁—, —O—, —S—, —CO—, —SO₂—. —CONR₁—, —CHOH—, —NR₁CO—, —SO₂NR₁—, —NR₁SO₂—, and wherein R₁ is independently hydrogen or alkyl of 1 to 6 carbon atoms; Rₕ′ and Rₕ″ are, independently from each other, alkyl groups from 2 to 18 carbon atoms; Rₕ is an alkyl group of 1 to 6 carbon atoms; x is 2 to 6; and m and n are 0 to 500 and m+n is equal to or greater than 1; and at least two hydrophilic units of Type 2, wherein each Type 2 unit is selected from the group consisting of formulas (2A), (2B) and (2C), wherein: L₄ is a divalent linking group, straight or branched, saturated or unsaturated hydrocarbon group of 1 to 10 carbon atoms or said hydrocarbon group interrupted by 1 to 3 groups selected from —NHR₁—, —O—, —S—, —CONR₁—, —NR₁CO—, —SO₂NR₁—, —NR₁SO₂— or terminated with —CO—, —SO₂— where the linking group L₄ is attached to the nitrogen in formula (2A) or (2B), and wherein R₁ is independently hydrogen or alkyl of 1 to 6 carbon atoms; L₅ is alkylene with 1 to 4 carbons; P₁ is a hydrophilic group and can be —COOH, —SO₃H, —PO₃H and salts thereof, —CONH₂, —CONHCH₂OH, or —(OCH₂CH₂)ₙOH; P₂ is —COO—; x is 2 to 6; p and q are 0 to 500; and r is 1 to 200.

The amino groups are substituted by a moiety further comprising: at least one alkyl or substituted alkyl unit of Type 3, wherein each Type 3 unit is selected from the group consisting of formulas (3A) and (3B), wherein: W₂ is a direct bond, —CO— or —SO2-; L₆ is a divalent linking group, either straight or branched alkylene group of 1 to 30 carbon atoms, or said alkylene group interrupted by 1 to 3 groups selected from the group consisting —NHR₁—, —O—, —S—, —CO—, —SO₂—. —CONR₁—, —CHOH—, —NR₁CO—, —SO₂NR₁—, —NR₁SO₂—, wherein R₁ is independently hydrogen or alkyl of 1 to 6 carbon atoms; and Q is hydrogen or a siloxane moiety selected from the group consisting of:

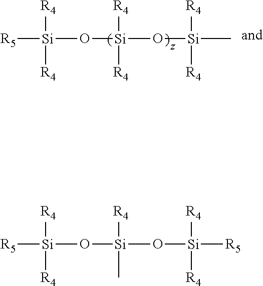

wherein R₄ and R₅ are independently lower alkyls of 1 to 6 carbon atoms and z is 1 to 100; x is 2 to 6; and s and t are 0 to 200 and s+t is equal or greater than 0.

Alternatively, the amino groups in such method may be substituted by a moiety comprising: at least two twin hydrocarbon chain units of Type 1, wherein each Type 1 unit is selected from the group consisting of formulas (1A) and (1B), wherein Rₕ′ and Rₕ″; are alkyl groups from six to twelve carbon atoms, W₁ is —CO—; L₁ is —CH₂CONH(CH₂)₃N⁺(CH₃)₂CH₂— or —CH₂—; L₂ and L₃ are, independently of each other, —CONH(CH₂)₂—, or —SO₂NH(CH₂)₂—; x is 2 or 3; n and m are 0 to 200; m+n is 2 to 200; and Rₕ is methyl; and at least two hydrophilic units of Type 2, wherein each Type 2 unit is selected from the group consisting of formulas (2A), (2B) and (2C), wherein L₄ is —CH₂—, —CH₂CH₂—, —CH═CH—, or —CH₂CH₂CONHC(CH₃)₂CH₂—; P₁ is —COOH, —SO₃H, or —PO₃H and salts thereof, x is 2, and p, q, and r are 1 to 100. The amino groups may be substituted by a moiety further comprising: at least one alkyl or substituted alkyl units of Type 3, wherein each Type 3 unit is selected from the group consisting of formulas (3A) and (3B), wherein x is 2, W₂ is —CO—, L₆ is a straight or branched alkylene group of 8 to 18 carbons, and Q is hydrogen.

The additive in such method may be derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA). The additive may also be derived from at least one polyethyleneimine, wherein the at least one polyethyleneimine has a molecular weight between about 300 and 100,000.

### EXAMPLES

### Example 1

An amido modified polyethyleneimine of formula [I-a] was synthesized according to the following reaction scheme:

84CH₃(CH₂)₇CH₂CO₂CH₃+(C₂H₅N)₅₈₀→CH₃(CH₂)₇CH₂CONH-modified PEI+352ClCH₂CO₂Na→CH₃(CH₂)₇CH₂CONH— and —NCH₂CO₂Na modified PEI [I-a]

A polyethyleneimine with average molecular weight of 25,000 (30 g, 1.2 mmole), propylene glycol (70 g), and methyl decanoate (18.63 g, 0.100 mole) were charged to a 250 mL Erlenmeyer flask equipped with a magnetic spin bar. The reaction mass was heated with agitation at 80° C. for one hour. Potassium tert-butoxide (5.6 g, 0.050 mole) was added to the flask and the reaction mass was heated with agitation at 80° C. for an additional 18 hours. FTIR analysis indicated that the amide formation was complete and that the ester band at 1725-1735 cm⁻¹ had disappeared. The temperature of the reaction mass was lowered to 70° C., sodium chloroacetate (51 g, 0.438 mole) was added over 90 minutes and the reaction mass was stirred for one hour. Propylene glycol (30 g) were then added and the reaction mass was stirred for 18 hours. The pH was adjusted with 50% NaOH to 7.2-7.5 and the reaction mass was diluted with distilled water (77 g) to yield a reddish-brown solution, (339.9 g) comprising 20.8% actives of the modified PEI of formula [I-a].

### Example 2

An amido modified polyethyleneimine of formula [II-a] comprising 20% actives was synthesized according to the following reaction scheme, and following the procedure outlined in example 1 for the modified PEI of formula [I-a].

105CH₃(CH₂)₅CH₂CO₂CH₃+(C₂H₅N)₅₈₀→CH₃(CH₂)₅CH₂CONH-modified PEI+331ClCH₂CO₂Na→CH₃(CH₂)₅CH₂CONH— and —NCH₂CO₂Na modified PEI [II-a]

### Example 3

An amido modified polyethyleneimine of formula [II-b] was synthesized according to the following reaction scheme, and following the procedure outlined in example 1 for the modified PEI of formula [I-a].

63CH₃(CH₂)₉CH₂CO₂CH₃+(C₂H₅N)₅₈₀→CH₃(CH₂)₉CH₂CONH-modified PEI+373ClCH₂CO₂Na→CH₃(CH₂)₉CH₂CONH— and —NCH₂CO₂Na modified PEI [II-b]

### Example 4

An amido modified polyethyleneimine of formula [III-a] was synthesized according to the following reaction scheme, and following the procedure outlined in example 1 for the modified PEI of formula [I-a].

84CH₃(CH₂)₉CH₂CO₂CH₃+(C₂H₅N)₅₈₀→CH₃(CH₂)₉CH₂CONH-modified PEI+352ClCH₂CO₂Na→CH₃(CH₂)₉CH₂CONH— and —NCH₂CO₂Na modified PEI [III-a]

### Example 5

A twin tail ester of formula [IV-a] comprising a cationic amino group was synthesized according to the following reaction scheme:

2CH₃(CH₂)₉CH₂CO₂CH₂CH₃+H₂NCH₂CH₂CH₂N(CH₃)CH₂CH₂CH₂NH₂→[CH₃(CH₂)₉CH₂CONHCH₂CH₂CH₂]2N(CH₃)+ClCH₂COOCH₃→[CH₃(CH₂)₉CH₂CONHCH₂CH₂CH₂]2N⁺(CH₃)CH₂COOCH₃ [IV-a]

3,3′-diamino-N-methyldipropylamine (9.08 g, 0.0625 mole), ethyl laurate (28.55 g, 0.125 mole) and propylene glycol (30 g) were charged into a 125 mL Erlenmeyer flask and agitated for 30 minutes using a magnetic spin bar. Potassium tert-butoxide (7.0 g, 0.0624 mole) was added to the flask and the reaction mass was heated with agitation at 80° C. for 3.5 hours. FTIR analysis indicated that the amide formation was complete and that the ester band at 1725-1735 cm⁻¹ had disappeared. The temperature was lowered to 65° C., methyl chloroacetate (7.46 g, 0.0688 mole) was added in one portion, and the reaction mass was stirred for 18 hours. Propylene glycol (10 g) was added to yield a solid melting at 45-55° C. (269.4 g), comprising 43.2% of the active ingredient formula [IV-a] (0.71 milliequivalents of formula [IV-a] per gram) and this solid was used without further purification.

### Example 6

A twin tail ester of formula [V-a] comprising a cationic amino group, was synthesized according to the following reaction scheme, and following the procedure outlined in example 5 for the twin tail ester of formula [IV-a], to yield a solid melting at 45-55° C. and comprising 0.82 milliequivalents of formula [V-a] per gram.

2CH₃(CH₂)₇CH₂CO₂CH₂CH₃+H₂NCH₂CH₂CH₂N(CH₃)CH₂CH₂CH₂NH₂→[CH₃(CH₂)₇CH₂CONHCH₂CH₂CH₂]2N(CH₃)+ClCH₂COOCH₃→[CH₃(CH₂)₇CH₂CONHCH₂CH₂CH₂]2N⁺(CH₃)CH₂COOCH₃ [V-a]

### Example 7

A twin tail ester of formula [VI-a] comprising a cationic amino group, was synthesized according to the following reaction scheme, and following the procedure outlined in example 5 for the twin tail ester of formula [IV-a], to yield a solid melting at 45-55° C. and comprising 0.84 milliequivalents of formula [VI-a] per gram.

2CH₃(CH₂)₅CH₂CO₂CH₂CH₃+H₂NCH₂CH₂CH₂N(CH₃)CH₂CH₂CH₂NH₂→[CH₃(CH₂)₅CH₂CONHCH₂CH₂CH₂]2N(CH₃)+ClCH₂COOCH₃→[CH₃(CH₂)₅CH₂CONHCH₂CH₂CH₂]₂N⁺(CH₃)CH₂COOCH₃ [VI-a]

### Example 8

A modified polyethyleneimine of formula [VII-a] comprising twin hydrocarbon tails and cationic amino groups was synthesized according to the following reaction scheme:

42[CH₃(CH₂)₉CH₂CONHCH₂CH₂CH₂]2N⁺(CH₃)CH₂COOCH₃+(C₂H₅N)₅₈₀→[CH₃(CH₂)₉CH₂CONHCH₂CH₂CH₂]2N⁺(CH₃)CH₂CONH-modified PEI+394ClCH₂CO₂Na→[CH₃(CH₂)₉CH₂CONHCH₂CH₂CH₂]2N⁺(CH₃)CH₂CONH— and —NCH₂CO₂Na modified PEI [VII-a]

A polyethyleneimine with average molecular weight of 25,000 (20 g, 0.8 mmole), propylene glycol (50 g), and the formula [IV-a] ester solution described in example 5 (46.99 g, 0.0333 mole) were charged to a 500 mL Erlenmeyer flask equipped with a magnetic spin bar. The reaction mass was heated with agitation at 80° C. for one hour. Potassium tert-butoxide (1.9 g, 0.0169 mole) was added to the flask and the reaction mass was heated with agitation at 80° C. for an additional three hours. FTIR analysis indicated that the amide formation was complete and that the ester band at 1725-1735 cm⁻¹ had disappeared. The temperature of the reaction mass was lowered to 70° C., sodium chloroacetate (19 g, 0.165 mole) was added and the reaction mass was stirred for one hour. Sodium chloroacetate (19 g, 0.165 mole) and propylene glycol (30 g) were then added and the reaction mass was stirred for one hour, at which point isopropyl alcohol (10 g) was added and stirring was continued for one hour. Propylene glycol (20 g) was added and stirring was continued for 15 hours. The pH was adjusted with 50% NaOH to 7.2-7.5 and the reaction mass was diluted with distilled water (61 g) to yield a reddish-brown solution comprising 21.1% actives of a modified PEI of formula [VII-a]. A precipitate formed upon cooling overnight to room temperature.

### Example 9

A modified polyethyleneimine of formula [VIII-a] comprising twin hydrocarbon tails and cationic amino groups, was synthesized according to the following reaction scheme as a solution comprising 21.2% actives, and following the procedure outlined in example 8 for the modified PEI of formula [VII-a].

52[CH₃(CH₂)₇CH₂CONHCH₂CH₂CH₂]2N⁺(CH₃)CH₂COOCH₃+(C₂H₅N)₅₈₀→[CH₃(CH₂)₇CH₂CONHCH₂CH₂CH₂]₂N⁺(CH₃)CH₂CONH-modified PEI+384ClCH₂CO₂Na→[CH₃(CH₂)₇CH₂CONHCH₂CH₂CH₂]2N⁺(CH₃)CH₂CONH— and —NCH₂CO₂Na modified PEI [VIII-a]

### Example 10

A modified polyethyleneimine of formula [IX-a] comprising twin hydrocarbon tails and cationic amino groups was synthesized according to the following reaction scheme as a solution comprising 21.4% actives, and following the procedure outlined in example 8 for the modified PEI of formula [VII-a].

63[CH₃(CH₂)₅CH₂CONHCH₂CH₂CH₂]2N⁺(CH₃)CH₂COOCH₃+(C₂H₅N)₅₈₀→[CH₃(CH₂)₅CH₂CONHCH₂CH₂CH₂]2N⁺(CH₃)CH₂CONH-modified PEI+373ClCH₂CO₂Na→[CH₃(CH₂)₅CH₂CONHCH₂CH₂CH₂]2N⁺(CH₃)CH₂CONH— and —NCH₂CO₂Na modified PEI [IX-a]

### Example 11

A polyethyleneimine modified with amido groups and comprising twin hydrocarbon tails and cationic amino groups was synthesized according to the following reaction scheme:

20CH₃(CH₂)₅CH₂CO₂CH₃+25[CH₃(CH₂)₅CH₂CONHCH₂CH₂CH₂]2N⁺(CH₃)CH₂COOCH₃+(C₂H₅N)₂₃₂→CH₃(CH₂)₇CH₂CON-modified and [CH₃(CH₂)₅CH₂CONHCH₂CH₂CH₂]₂N⁺(CH₃)CH₂CONH-modified PEI+130ClCH₂CO₂Na→CH₃(CH₂)₅CH₂CON—,[CH₃(CH₂)₅CH₂CONHCH₂CH₂CH₂]2N⁺(CH₃)CH₂CONH—, and —NCH₂CO₂Na modified PEI [X-a]

A polyethyleneimine with average molecular weight 10,000 (5 g, 0.5 mmole), propylene glycol (15 g), methyl octoate (18.60 g, 0.100 mole), and the twin tail ester solution described in example 7 (14.49 g, 0.0125 mole) were charged to a 250 mL Erlenmeyer flask equipped with a magnetic spin bar. The reaction mass was heated with agitation at 80° C. for one hour. Potassium tert-butoxide (0.46 g, 4.10 mmole) was added to the flask and the reaction mass was heated with agitation at 80° C. for an additional 15 hours. Potassium tert-butoxide (0.5 g, 4.46 mmole) was added to the flask over five hours while the reaction mass was heated with agitation at 90° C. FTIR analysis indicated that the amide formation was complete and that the ester band at 1725-1735 cm⁻¹ had disappeared. The temperature of the reaction mass was lowered to 70° C., sodium chloroacetate (8.26 g, 0.0709 mole) and propylene glycol (10 g) were then added and the reaction mass was stirred for 15 hours. The pH was adjusted with 50% NaOH to 7.2-7.5 and the reaction mass was diluted with distilled water (15 g) to yield a reddish-brown solution, (67.7 g) comprising 23% actives of a modified PEI of formula [X-a].

### Example 12

A polyethyleneimine modified with amido groups and comprising twin hydrocarbon tails and cationic amino groups, was synthesized according to the following reaction scheme as a solution comprising 23.3% actives, utilizing the twin tail ester described in example 6 and following the procedure outlined in example 11 for the modified PEI of formula [X-a].

8CH₃(CH₂)₇CH₂CO₂CH₃+17[CH₃(CH₂)₇CH₂CONHCH₂CH₂CH₂]2N⁺(CH₃)CH₂COOCH₃+(C₂H₅N)₂₃₂→CH₃(CH₂)₇CH₂CONH-modified and [CH₃(CH₂)₇CH₂CONHCH₂CH₂CH₂]2N⁺(CH₃)CH₂CONH-modified PEI+150ClCH₂CO₂Na→CH₃(CH₂)₇CH₂CON—,[CH₃(CH₂)₇CH₂CONHCH₂CH₂CH₂]₂N⁺(CH₃)CH₂CONH—, and —NCH₂CO₂Na modified PEI [XI-a]

### Example 13

A polyethyleneimine modified with amido groups and comprising twin hydrocarbon tails and cationic amino groups, was synthesized according to the following reaction scheme as a solution comprising 21.2% actives, utilizing the twin tail ester described in example 6 and following the procedure outlined in example 11 for the modified PEI of formula [X-a].

17CH₃(CH₂)₇CH₂CO₂CH₃+8[CH₃(CH₂)₇CH₂CONHCH₂CH₂CH₂]2N⁺(CH₃)CH₂COOCH₃+(C₂H₅N)₂₃₂→CH₃(CH₂)₇CH₂CONH-modified and [CH₃(CH₂)₇CH₂CONHCH₂CH₂CH₂]2N⁺(CH₃)CH₂CONH-modified PEI+150ClCH₂CO₂Na→CH₃(CH₂)₇CH₂CONH—,[CH₃(CH₂)₇CH₂CONHCH₂CH₂CH₂]2N⁺(CH₃)CH₂CONH—, and —NCH₂CO₂Na modified PEI [XII-a]

### Example 14

A polyethyleneimine modified with amido groups and comprising twin hydrocarbon tails and cationic amino groups, was synthesized according to the following reaction scheme as a solution comprising 22.3% actives, utilizing the twin tail ester described in example 7 and following the procedure outlined in example 11 for the modified PEI of formula [X-a].

17CH₃(CH₂)₅CH₂CO₂CH₃+21[CH₃(CH₂)₅CH₂CONHCH₂CH₂CH₂]2N⁺(CH₃)CH₂COOCH₃+(C₂H₅N)₂₃₂→CH₃(CH₂)₇CH₂CONH-modified and [CH₃(CH₂)₅CH₂CONHCH₂CH₂CH₂]2N⁺(CH₃)CH₂CONH-modified PEI+137ClCH₂CO₂Na→CH₃(CH₂)₅CH₂CONH—,[CH₃(CH₂)₅CH₂CONHCH₂CH₂CH₂]2N⁺(CH₃)CH₂CONH—, and —NCH₂CO₂Na modified PEI [XIII-a]

### Example 15

A modified polyethyleneimine of formula [XIV-a] comprising twin hydrocarbon tails and cationic amino groups could be synthesized according to the following reaction scheme, and following the procedure outlined in example 8 for the modified PEI of formula [VII-a].

52[CH₃(CH₂)₇CH₂CONHCH₂CH₂CH₂]2N⁺(CH₃)CH₂COOCH₃+(C₂H₅N)₅₈₀→[CH₃(CH₂)₇CH₂CONHCH₂CH₂CH₂]2N⁺(CH₃)CH₂CONH-modified PEI+384CH₂═CHCO₂Na→[CH₃(CH₂)₇CH₂CONHCH₂CH₂CH₂]2N⁺(CH₃)CH₂CON— and —NCH₂CH₂CO₂Na modified PEI [XIV-a]

### Example 16

A modified polyethyleneimine of formula [XV-a] comprising twin hydrocarbon tails and cationic amino groups could be synthesized according to the following reaction scheme, and following the procedure outlined in example 8 for the modified PEI of formula [VII-a].

3[CH₃(CH₂)₇CH₂CONHCH₂CH₂CH₂]2N⁺(CH₃)CH₂COOCH₃+(C₂H₅N)₂₈→[CH₃(CH₂)₇CH₂CONHCH₂CH₂CH₂]2N⁺(CH₃)CH₂CON-modified PEI+18CH₂═CHCO₂Na→[CH₃(CH₂)₇CH₂CONHCH₂CH₂CH₂]2N⁺(CH₃)CH₂CON— and —NCH₂CH₂CO₂Na modified PEI [XV-a]

### Example 17

Table 1 is comparative and illustrates that the presence of twin tail cationic side chains as in the compounds of the invention, is far more effective in lowering surface tension than the presence of a comparable concentration of single hydrocarbon chains of the same length. Surface tension measurements were recorded using a Kruss model FM40Mk2 instrument for solutions of the modified PEI's comprising 0.1% actives in deionized water.

TABLE 1
Impact of twin-tail cationic side chains on surface tension

| Modified PEI from example | Surface tension (dyn/cm) |
|---|---|
| 2 | 32.6 |
| 10 | 25.4 |

### Example 18

Table 2 is comparative and illustrates that the presence of twin tail cationic side chains as in the compounds of the invention, is far more effective in lowering surface tension than the presence of a comparable concentration of single hydrocarbon chains of the same length. Surface tension measurements were recorded using a Kruss model FM40Mk2 instrument for solutions of the modified PEI's comprising 0.1% actives in deionized water.

TABLE 2
Impact of twin-tail cationic side chains on surface tension

| Modified PEI from example | Surface tension (dyn/cm) |
|---|---|
| 9 | 30.0 |
| 10 | 23.1 |

### Example 19

Table 3 is comparative and illustrates that the presence of twin tail cationic side chains as in the compounds of the invention, is far more effective in lowering surface tension than the presence of a comparable concentration of single hydrocarbon chains of the same length. Surface tension measurements were recorded using a Kruss model FM40Mk2 instrument for solutions of the modified PEI's comprising 0.1% actives in deionized water.

TABLE 3
Impact of twin-tail cationic side chains on surface tension

| Modified PEI from example | Surface tension (dyn/cm) |
|---|---|
| 4 | 28.5 |
| 8 | 22.1 |

### Example 20

Table 4 is comparative and illustrates that the presence of cationic group comprising twin tail hydrocarbon side chains in a modified PEI as in the compounds of the invention, is far more effective in lowering surface tension than the presence of an equal concentration of single hydrocarbon chains of the same length. Surface tension measurements were recorded using a Kruss model FM40Mk2 instrument for solutions of the modified PEI's comprising 0.1% actives in deionized water.

TABLE 4
Impact of twin-tail cationic side chains on surface tension

| | | Number of | Number of | |
|---|---|---|---|---|
| Modified | Total number of | twin tail | single tail | Surface |
| PEI from | C₁₀ amide | hydrocarbon | hydrocarbon | tension |
| example | side chains * | groups | groups | (dyn/cm) |
| 1 | 4 | 0 | 4 | 30.8 |
| 13 | 4 | 1 | 2 | 22.7 |
| 12 | 4 | 2 | 0 | 22.7 |

* Per 1200 g of PEI starting material

The compositions and methods disclosed herein for foam compositions provide significant benefits compared with conventional foam compositions. Conventional foam compositions comprise C6 fluorochemicals, which are anticipated to become regulated by governments in the near future as a result of environmental concerns. The present compositions and methods, however, disclose novel water soluble oligomeric and polymeric additives derived from oligomeric and polymeric amines; esters or halides of twin tail alkyl group comprising acids with intervening quaternary ammonium groups; optionally and preferably hydrophilic group comprising compounds capable of reacting with primary, secondary or tertiary amino groups; and optionally hydrophobic group comprising compounds, can perform as partial or complete replacements for fluorinated foam stabilizers and fluorosurfactants in foam formulations. Very unexpectedly, the inventors determined that inclusion of twin alkyl tails and quaternary ammonium intervening groups lowers the surface tension to levels where vapor suppression can occur. The instant additives allow the formulation of foam agents with low levels of fluorosurfactants and fluorinated foam stabilizers, and in selected cases allow the formulation of fluorochemical-free agents. This partial or complete replacement of the fluorosurfactants and fluorinated foam stabilizers results in the formulation of lower cost agents, as well as in lower release of fluorosurfactants into the environment.

The compositions disclosed herein are useful for several commercial applications, including but not limited to partial or complete replacements for fluorosurfactants and/or fluorinated foam stabilizers in firefighting foam formulations. Certain foams, for example, are ineffective in fighting fires caused by polar solvents such as alcohols, because the foam is destroyed by mixing with the water-miscible solvent; the presently disclosed additives, however, allow the formulation of firefighting foam agents with low levels of fluorosurfactants and fluorinated foam stabilizers, and in selected cases allow the formulation of fluorochemical-free agents. The presently disclosure, therefore, contemplates fire extinguishers and other fire extinguishing systems configured to deliver each of the compositions disclosed herein.

This application references various publications. The disclosures of these publications, in their entireties, are hereby incorporated by reference into this application to describe more fully the state of the art to which this application pertains. The references disclosed are also individually and specifically incorporated herein by reference for material contained within them that is discussed in the sentence in which the reference is relied on.

The embodiments of the compositions and methodologies described herein are exemplary. Various other embodiments of the compositions and methodologies described herein are possible.

Now, therefore, the following is claimed:

1. A composition comprising:
a polyamine with a molecular weight between about 103 and 100,000;
wherein the amino groups are substituted by a moiety comprising: at least one twin tail alkyl group; at least one quaternary ammonium group; and at least one non-amino hydrophilic group.

2. The composition of claim 1, wherein the moiety further comprises at least one hydrophobic group.

3. The composition of claim 1, wherein the amino groups are partially substituted.

4. The composition of claim 1, wherein the amino groups are fully substituted.

5. The composition of claim 1, wherein the amino groups are substituted by a moiety comprising:
at least two twin hydrocarbon chain units of Type 1, wherein each unit of Type 1 is selected from the group consisting of formulas (1A) and (1B): 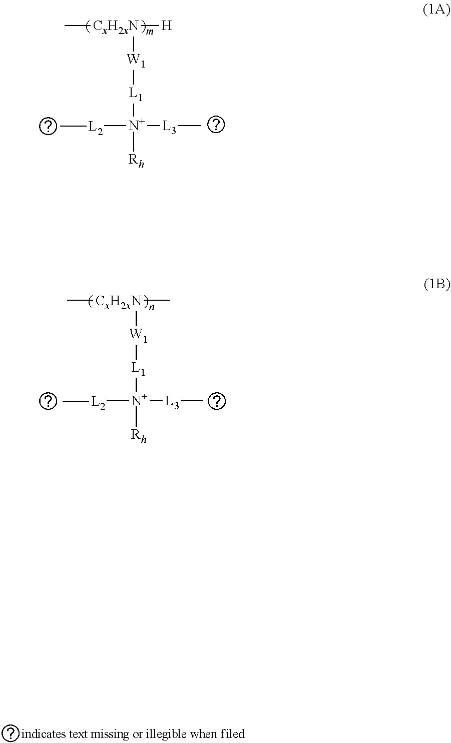
wherein:
W₁ is —CO— or —SO2-;
L₁ is independently a divalent linking group, either straight or branched alkylene group of 1 to 15 carbon atoms, or said alkylene group interrupted by 1 to 5 groups selected from the group consisting of —NHR₁—, —O—, —S—, —CO—, —SO₂—. —CONR₁—, —CHOH—, —NR₁CO—, —SO₂NR₁—, —NR₁SO₂—, —R₂N⁺R₃—, wherein R₁ is independently hydrogen or alkyl of 1 to 6 carbon atoms and R₂ and R₃ are independently alkyl groups of 1 to 6 carbon atoms;
L₂, and L₃ are, independently of each other, divalent linking groups, either straight or branched alkylene groups of 1 to 10 carbon atoms, or said alkylene groups interrupted by 1 to 3 groups selected from the group consisting of —NHR₁—, —O—, —S—, —CO—, —SO₂—. —CONR₁—, —CHOH—, —NR₁CO—, —SO₂NR₁—, —NR₁SO₂—, and wherein R₁ is independently hydrogen or alkyl of 1 to 6 carbon atoms;
Rₕ′ and Rₕ″ are, independently from each other, alkyl groups from 2 to 18 carbon atoms;
Rₕ is an alkyl group of 1 to 6 carbon atoms;
x is 2 to 6; and
m and n are 0 to 500 and m+n is equal to or greater than 1; and
at least two hydrophilic units of Type 2, wherein each unit of Type 2 is selected from the group consisting of formulas (2A), (2B) and (2C): 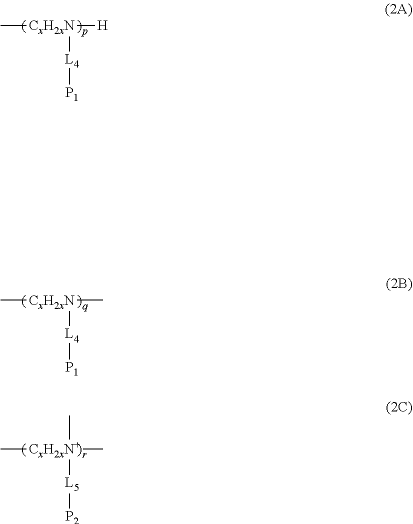
wherein:
L₄ is a divalent linking group, straight or branched, saturated or unsaturated hydrocarbon group of 1 to 10 carbon atoms or said hydrocarbon group interrupted by 1 to 3 groups selected from —NHR₁—, —O—, —S—, —CONR₁—, —NR₁CO—, —SO₂NR₁—, —NR₁SO₂— or terminated with —CO—, —SO₂— where the linking group L₄ is attached to the nitrogen in formula (2A) or (2B), and wherein R₁ is independently hydrogen or alkyl of 1 to 6 carbon atoms;
L₅ is alkylene with 1 to 4 carbons;
P₁ is a hydrophilic group and can be —COOH, —SO₃H, —PO₃H and salts thereof, —CONH₂, —CONHCH₂OH, or —(OCH₂CH₂)ₙOH;
P₂ is —COO⁻;
x is 2 to 6;
p and q are 0 to 500; and
r is 1 to 200.

6. The composition of claim 5, wherein the amino groups are further substituted by a moiety comprising:
at least one alkyl or substituted alkyl unit of Type 3, wherein each Type 3 unit is selected from the group consisting of formulas (3A) and (3B): 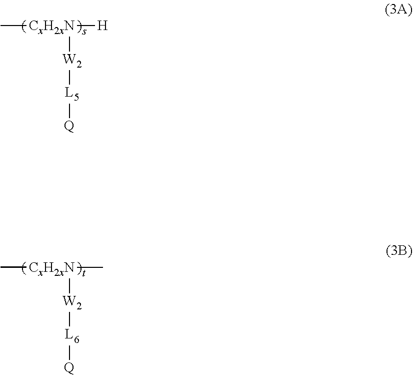
wherein:
W₂ is a direct bond, —CO— or —SO2-;
L₆ is a divalent linking group, either straight or branched alkylene group of 1 to 30 carbon atoms, or said alkylene group interrupted by 1 to 3 groups selected from the group consisting —NHR₁—, —O—, —S—, —CO—, —SO₂—. —CONR₁—, —CHOH—, —NR₁CO—, —SO₂NR₁—, —NR₁SO₂—, wherein R₁ is independently hydrogen or alkyl of 1 to 6 carbon atoms; and
Q is hydrogen or a siloxane moiety selected from the group consisting of: 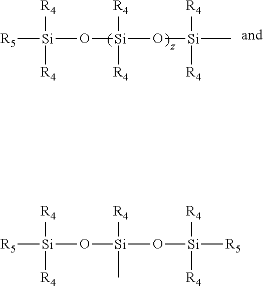
wherein R₄ and R₅ are independently lower alkyls of 1 to 6 carbon atoms and z is 1 to 100; x is 2 to 6; and s and t are 0 to 200 and s+t is equal or greater than 0.

7. The composition of claim 1, wherein the amino groups are substituted by a moiety comprising:
at least two twin hydrocarbon chain units of Type 1, wherein each Type 1 unit is selected from the group consisting of formulas (1A) and (1B): 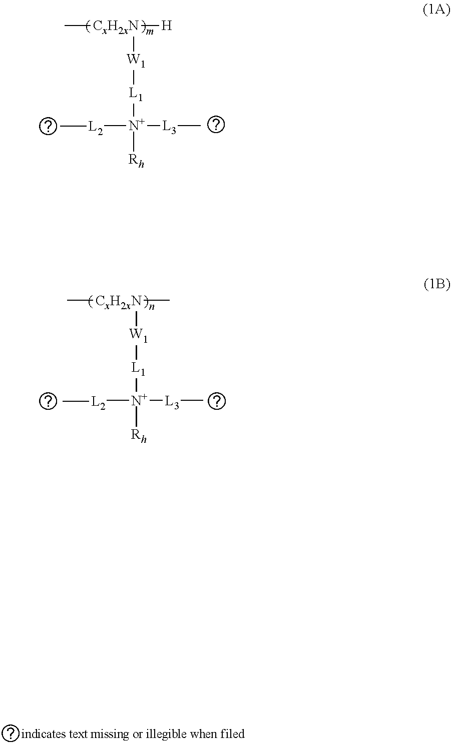
wherein Rₕ′ and Rₕ″; are alkyl groups from six to twelve carbon atoms, W₁ is —CO—; L₁ is —CH₂CONH(CH₂)₃N⁺(CH₃)₂CH₂— or —CH₂—; L₂ and L₃ are, independently of each other, —CONH(CH₂)₂—, or —SO₂NH(CH₂)₂—; x is 2 or 3; n and m are 0 to 200; m+n is 2 to 200; and Rₕ is methyl; and
at least two hydrophilic units of Type 2, wherein each Type 2 unit is selected from the group consisting of formulas (2A), (2B) and (2C): 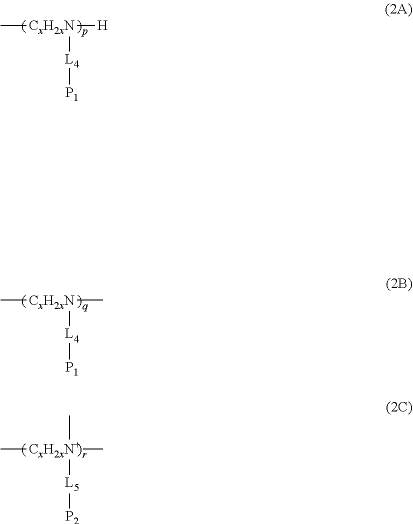
wherein L₄ is —CH₂—, —CH₂CH₂—, —CH═CH—, or —CH₂CH₂CONHC(CH₃)₂CH₂—; P₁ is —COOH, —SO₃H, or —PO₃H and salts thereof, x is 2, and p, q, and r are 1 to 100.

8. The composition of claim 7, wherein the amino groups are substituted by a moiety further comprising:
at least one alkyl or substituted alkyl unit of Type 3, wherein each Type 3 unit is selected from the group consisting of formulas (3A) and (3B): 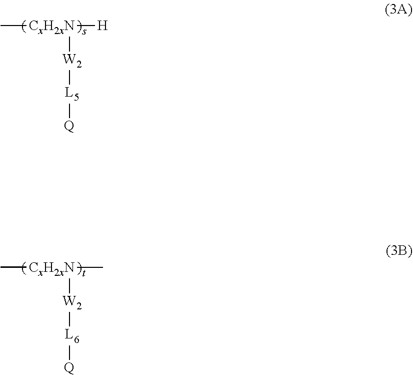
wherein x is 2, W₂ is —CO—, L₆ is a straight or branched alkylene group of 8 to 18 carbons, and Q is hydrogen.

9. The composition of claim 1, wherein the composition is derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA).

10. The composition of claim 1, wherein the composition is derived from at least one polyethyleneimine with a molecular weight between about 300 and 100,000.

11. A method of improving the stability of aqueous and alcohol-resistant foam compositions comprising the steps of:
adding an effective amount of a water soluble aqueous film forming foam additive to an agent;
wherein the additive comprises a polyamine with a molecular weight between about 103 and 100,000; and
wherein the amino groups are substituted by a moiety comprising: at least one twin tail alkyl group; at least one quaternary ammonium group; and at least one non-amino hydrophilic group.

12. The method of claim 11, wherein the amino groups are partially substituted.

13. The method of claim 11, wherein the amino groups are fully substituted.

14. The method of claim 11, wherein the amino groups are substituted by a moiety further comprising at least one hydrophobic group.

15. The method of claim 11, wherein the amino groups are substituted by a moiety comprising:
at least two twin hydrocarbon chain units of Type 1, wherein each Type 1 unit is selected from the group consisting of formulas (1A) and (1B): 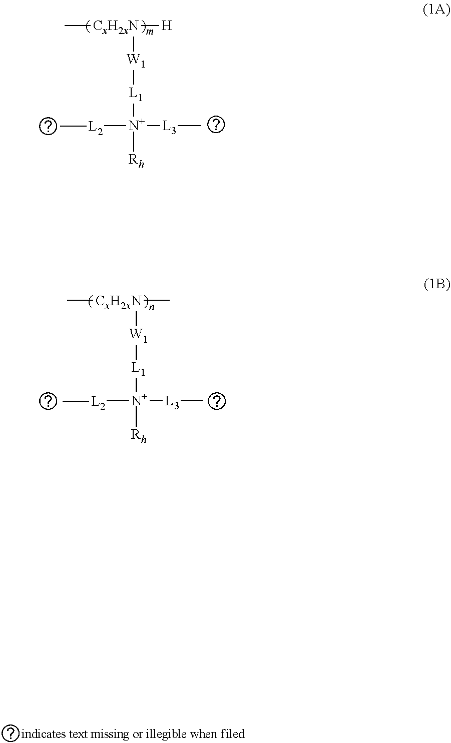
wherein:
W₁ is —CO— or —SO2-;
L₁ is independently a divalent linking group, either straight or branched alkylene group of 1 to 15 carbon atoms, or said alkylene group interrupted by 1 to 5 groups selected from the group consisting of —NHR₁—, —O—, —S—, —CO—, —SO₂—. —CONR₁—, —CHOH—, —NR₁CO—, —SO₂NR₁—, —NR₁SO₂—, —R₂N⁺R₃—, wherein R₁ is independently hydrogen or alkyl of 1 to 6 carbon atoms and R₂ and R₃ are independently alkyl groups of 1 to 6 carbon atoms;
L₂, and L₃ are, independently of each other, divalent linking groups, either straight or branched alkylene groups of 1 to 10 carbon atoms, or said alkylene groups interrupted by 1 to 3 groups selected from the group consisting of —NHR₁—, —O—, —S—, —CO—, —SO₂—. —CONR₁—, —CHOH—, —NR₁CO—, —SO₂NR₁—, —NR₁SO₂—, and wherein R₁ is independently hydrogen or alkyl of 1 to 6 carbon atoms;
Rₕ′ and Rₕ″ are, independently from each other, alkyl groups from 2 to 18 carbon atoms;
Rₕ is an alkyl group of 1 to 6 carbon atoms;
x is 2 to 6; and
m and n are 0 to 500 and m+n is equal to or greater than 1; and
at least two hydrophilic units of Type 2, wherein each Type 2 unit is selected from the group consisting of formulas (2A), (2B) and (2C): 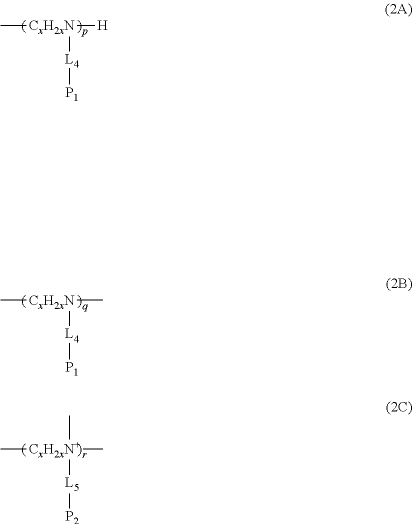
wherein:
L₄ is a divalent linking group, straight or branched, saturated or unsaturated hydrocarbon group of 1 to 10 carbon atoms or said hydrocarbon group interrupted by 1 to 3 groups selected from —NHR₁—, —O—, —S—, —CONR₁—, —NR₁CO—, —SO₂NR₁—, —NR₁SO₂— or terminated with —CO—, —SO₂— where the linking group L₄ is attached to the nitrogen in formula (2A) or (2B), and wherein R₁ is independently hydrogen or alkyl of 1 to 6 carbon atoms;
L₅ is alkylene with 1 to 4 carbons;
P₁ is a hydrophilic group and can be —COOH, —SO₃H, —PO₃H and salts thereof, —CONH₂, —CONHCH₂OH, or —(OCH₂CH₂)ₙOH;
P₂ is —COO⁻;
x is 2 to 6;
p and q are 0 to 500; and
r is 1 to 200.

16. The method of claim 15, wherein the amino groups are substituted by a moiety further comprising:
at least one alkyl or substituted alkyl unit of Type 3, wherein each Type 3 unit is selected from the group consisting of formulas (3A) and (3B): 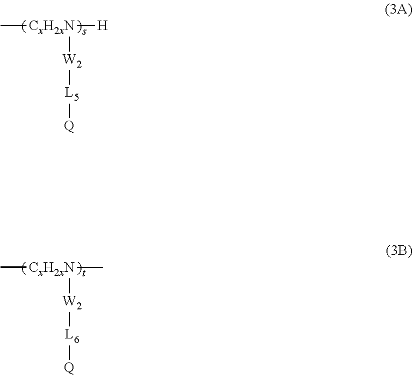
wherein:
W₂ is a direct bond, —CO— or —SO2-;
L₆ is a divalent linking group, either straight or branched alkylene group of 1 to 30 carbon atoms, or said alkylene group interrupted by 1 to 3 groups selected from the group consisting —NHR₁—, —O—, —S—, —CO—, —SO₂—. —CONR₁—, —CHOH—, —NR₁CO—, —SO₂NR₁—, —NR₁SO₂—, wherein R₁ is independently hydrogen or alkyl of 1 to 6 carbon atoms; and
Q is hydrogen or a siloxane moiety selected from the group consisting of: 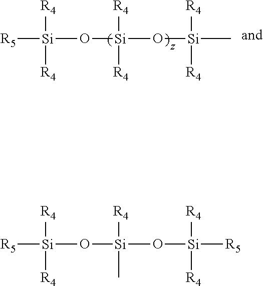
wherein R₄ and R₅ are independently lower alkyls of 1 to 6 carbon atoms and z is 1 to 100; x is 2 to 6; and s and t are 0 to 200 and s+t is equal or greater than 0.

17. The method of claim 11, wherein the amino groups are substituted by a moiety comprising:
at least two twin hydrocarbon chain units of Type 1, wherein each Type 1 unit is selected from the group consisting of formulas (1A) and (1B): 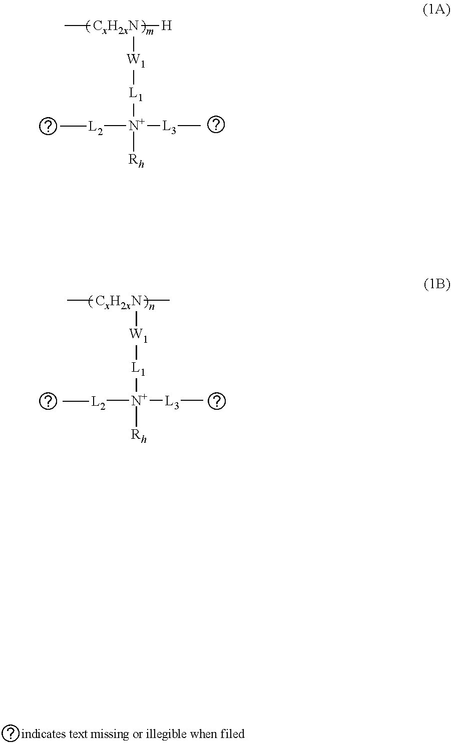
wherein Rₕ′ and Rₕ″; are alkyl groups from six to twelve carbon atoms, W₁ is —CO—; L₁ is —CH₂CONH(CH₂)₃N⁺(CH₃)₂CH₂— or —CH₂—; L₂ and L₃ are, independently of each other, —CONH(CH₂)₂—, or —SO₂NH(CH₂)₂—; x is 2 or 3; n and m are 0 to 200; m+n is 2 to 200; and Rₕ is methyl; and
at least two hydrophilic units of Type 2, wherein each Type 2 unit is selected from the group consisting of formulas (2A), (2B) and (2C): 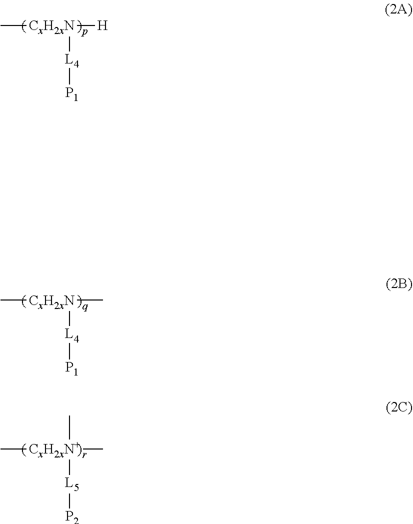
wherein L₄ is —CH₂—, —CH₂CH₂—, —CH═CH—, or —CH₂CH₂CONHC(CH₃)₂CH₂—; P₁ is —COOH, —SO₃H, or —PO₃H and salts thereof, x is 2, and p, q, and r are 1 to 100.

18. The method of claim 17, wherein the amino groups are substituted by a moiety further comprising:
at least one alkyl or substituted alkyl units of Type 3, wherein each Type 3 unit is selected from the group consisting of formulas (3A) and (3B): 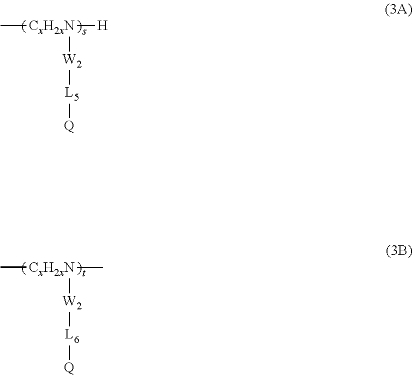
wherein x is 2, W₂ is —CO—, L₆ is a straight or branched alkylene group of 8 to 18 carbons, and Q is hydrogen.

19. The method of claim 11, wherein the additive is derived from diethylenetriamine (DETA), triethylenetetramine (TETA), tetraethylenepentamine (TEPA), pentaethylene hexamine (PEHA), aminoethylpiperazine (AEP), or iminobispropylamine (IBPA).

20. The method of claim 11, wherein the additive is derived from at least one polyethyleneimine, wherein the at least one polyethyleneimine has a molecular weight between about 300 and 100,000.
